(19) United States
(12) Patent Application Publication
(10) Pub. No.: US 2006/0062812 A1
(43) Pub. Date: Mar. 23, 2006

(54) Novel compositions

(76) Inventors: Calvin Ross, Essex (GB); Clive Booles, Essex (GB); Alistair Campbell, Essex (GB); Paul William Woodcock, Essex (GB); Scott Andrew Davis, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/224,383

(22) Filed: Sep. 12, 2005

Related U.S. Application Data
(63) Continuation-in-part of application No. PCT/GB04/01037, filed on Mar. 11, 2004.

(30) Foreign Application Priority Data
Mar. 11, 2003 (GB) 0305579.5
Dec. 3, 2003 (GB) 0328023.7
Sep. 10, 2004 (GB) 0420173.7

(51) Int. Cl. A61K 31/445; A61K 31/045; A61K 9/00

(52) U.S. Cl. 424/400; 514/317; 514/729

(57) ABSTRACT

Compositions and method are provided, for the treatment of pain, e.g. acute breakthrough pain, by means of a systemic, non-invasive mode of administration. Specifically, the invention relates to a sublingual presentation of an opioid analgesic, such as fentanyl, or its salts, in amounts that are sufficient to treat the pain.

### CROSS-REFERENCE TO EARLIER APPLICATION

This application is a continuation-in-part application of International Application PCT/GB04/01037, filed Mar. 11, 2004; which claims priority to GB 0305579.5, filed Mar. 11, 2003 and GB 0328023.7, filed Dec. 3, 2003. The subject application further claims priority to GB 0420173.7, filed Sep. 10, 2004, all of which are hereby incorporated by reference herein in their entirety.

### FIELD OF THE INVENTION

This invention relates to formulations of opioid analgesics and in particular fentanyl, especially pump spray formulations suitable for sublingual delivery.

### BACKGROUND OF THE INVENTION

Opioid analgesics are useful in the treatment of pain, such as breakthrough pain. When treating pain, it is particularly attractive for the patient to be able to self-medicate, enabling specific pain episodes to be treated, as opposed to ongoing treatment when there may be no pain to treat. It is highly desirable for the onset of analgesia to occur as soon after administration of the opioid analgesic as possible, especially where the patient is self-medicating. This not only provides pain relief as soon as possible but it can also reduce the risk of overdosage. A delay in the onset of the therapeutic effect may prompt the patient to take a further dose, with the consequent risk of the serious side-effects associated with overdosage.

In the case of breakthrough pain, the onset of pain is relatively quick, usually between just a few seconds and 10 to 15 minutes, the median being approximately 3 minutes. The duration of breakthrough pain episodes tends to be anywhere between 5 minutes and 2 hours, the median being between 20 and 60 minutes.

Thus, for the most effective treatment of pain, and in particular, breakthrough pain, the analgesic effect should have a rapid onset. What is more, the analgesic effect should last for the duration of the pain episode. That said, administration of a second or further dose may be acceptable, provided that these additional doses have a rapid onset of effect, so that the pain is not left untreated for too long.

Fentanyl is a narcotic alkaloid, which has been used for many years as an anaesthetic and an analgesic, especially in the treatment of moderate to severe pain. Whilst undoubtedly effective for pain relief, and especially in the treatment of pain which is refractive to other treatments, there are a number of issues of clinical management associated with the use of fentanyl in therapy.

Foremost amongst these issues is the potential for serious side-effects with fentanyl. It has a much higher potency than commonly known narcotics and therefore it is necessary to ensure that it is being used within the established therapeutically effective range and to monitor patients for evidence of self-medication at greater than the recommended amount. Overdosage with fentanyl can lead to a number of undesirable and indeed life-threatening side-effects, predominantly hypoventilation and respiratory depression.

A number of routes of administration of a medicament can be associated with rapid onset of action. For example, WO90/07333 describes aerosol formulations of fentanyl, which are adapted for inhalation. However, these formulations suffer disadvantages such as their use of hydrofluorocarbon propellants and delivery effected by metered dose inhalers. In the case of the former, the disadvantages include high velocity which results in “bounce-back” on administration to the front of the mouth, cold sensations on administration and the risk of inhalation; for the latter, careful co-ordination of breath and actuation by the patient. When metered dose inhalers are used, a significant proportion of the delivered dose tends to impact the back of the throat from where it is swallowed rather than finding its way into the bronchial passages. Accordingly, the pharmacology of the medication may be unpredictable due to poor bioavailability following oral administration or may be characterised by a bi-phasic profile (fast initial onset as a result of the inhaled dose and a slower, late effect due to oral absorption of fentanyl). Furthermore, manufacture of the bulk formulation involves the preparation of large quantities of pressurised volatile propellant containing a potent narcotic analgesic. Accordingly, the precautions required to ensure safe manufacture are onerous and expensive.

WO95/31182 describes solution formulations of fentanyl in aerosol propellants intended for administration to patients by the pulmonary route.

WO01/97780 describes solution formulations of fentanyl free base in propellants, typically HFA134a, for sublingual aerosol administration.

WO00/47203 describes formulations of fentanyl citrate for intra-oral administration employing oral absorption enhancers.

Certain aqueous formulations of fentanyl for intranasal administration employing water and phosphate buffer have been described; see Paech, M. J., Lim, C. B., Banks, S. L., Rucklidge, M. W. M. & Doherty, D. A. (2003) Anaesthesia 58 (8), 740-744, and Lim et al (2003) J Pharm Practice Research 33, 59-63. Such formulations can suffer problems of nasal irritation associated with medium to long term usage via this route which is undesirable. Weinberg et al (1988) Clin Pharmacol Therap 44 335-342, discloses formulations of fentanyl employing water and phosphate buffer for sublingual administration, but these formulations are not advocated for use as a spray.

It is well known that the application of carefully chosen medicaments to the sublingual mucosa offers a route of administration which is capable of resulting in very rapid transmission of medicament to the bloodstream with consequent fast onset of effect. A number of ways of administering compositions sublingually are known. For example, tablets or liquids may be held under the tongue prior to swallowing. Another method is spray delivery. Of these various types of sublingual administration, spray delivery is preferred as it does not involve holding the composition under the tongue for an extended period of time as, for example, with a lozenge, and it reduces the amount of material which is swallowed (and may enter the blood stream in a delayed manner via the gastrointestinal tract). Pharmaceutical compositions, for example a fentanyl lozenge, cause increased salivation, which facilitates the unwanted swallowing of drug substance.

In the past, spray devices, including pump sprays, have been proposed for sublingual administration. However, their effect has not been properly optimised. In order to reduce the amount of the dispensed composition which fails to contact the sublingual mucosa, the compositions tend to be dispensed in a focussed manner, so that the sublingual spray devices have a tendency to administer the compositions to a relatively small part of the sublingual mucosa. This means that the composition is effectively concentrated in the relatively small area, which slows down absorption and also means that some of the composition may not be absorbed, but rather may be washed away by saliva and swallowed. This is a particular problem in the case of lipophilic opioid analgesics such as fentanyl. It has been shown that the lipophilic drugs need to be finely spread over the sublingual mucosa in order for them to be properly absorbed. When they are concentrated at a small area of the sublingual mucosa, absorption is reduced.

It is an aim of the present invention to provide a formulation, which avoids or mitigates some or all of the above-mentioned disadvantages.

Another aim of the present invention is to provide a presentation of an opioid analgesic for treating pain, and in particular breakthrough pain, wherein the opioid analgesic is administered via the sublingual route and the presentation preferably exhibits improved performance compared to known opioid analgesic compositions, including those which may be administered sublingually and intravenously. In particular, it is an aim of the invention to provide fast onset of therapeutic effect, together with an advantageous pharmacokinetic response and drug plasma profile which will avoid the disadvantages associated with the fast onset observed when opioid analgesics are administered intravenously.

### SUMMARY OF THE INVENTION

The present invention is based at least in part on the understanding that spray delivery, having low volume and ability to target the sublingual mucosa, largely mitigates problems associated with other formulations, and can avoid the use of propellants.

According to the invention, a pharmaceutical composition, preferably a partially pressurised liquid spray formulation, comprises:
(a) fentanyl or a pharmaceutically acceptable salt thereof;
(b) water as carrier; and
(c) a polar organic solvent in sufficient amount to enhance the solubility of the fentanyl or pharmaceutically acceptable salt thereof in the water.

The formulations of the invention may be used in analgesia and for the treatment of pain. They are preferably administered sublingually as a spray. The formulations are well tolerated when administered to the sensitive sublingual mucosa and the sublingual spray administration will result in rapid onset of the therapeutic effect of the fentanyl.

The present invention also provides a pharmaceutical composition for use in the treatment of acute breakthrough pain by means of a systemic, non-invasive mode of administration. Specifically, the invention relates to a sublingual presentation of an opioid analgesic, such as fentanyl, or its salts, in amounts that are sufficient to treat the acute pain. Advantageously, the presentation of the opioid analgesic provides a rapid onset of action, as well as a pharmacokinetic response and drug plasma profile suitable to achieve optimal pain relief over the duration of symptoms with minimized side-effects.

The invention also relates to a specific drug formulation, dispensed using a metered pump action spray which is specifically designed for delivery via the sublingual route. This affords significant improvements and advantages in terms of plasma bioavailability and pharmacokinetic profile compared to similar, but non-optimised, propellant-driven aerosol formulations. These benefits relate in particular to:
i) a faster rate of onset of effect;
ii) a faster rate of offset of effect; and
iii) a faster Tmax.

According to a further aspect of the invention, an opioid analgesic pharmaceutical composition provides an opioid analgesic plasma concentration of 250 pg/ml within a period of no more than 2 hours, following sublingual administration using a pump spray dispensing device. The opioid analgesic is preferably fentanyl.

Amongst the advantages of these formulations is the fact that, by being water-based, they avoid the issues associated with using pressurised hydrofluorocarbon propellants as mentioned above. The formulations may be partially pressurised and are free of propellants such as volatile chlorofluorocarbons (e.g. propellant 12), volatile hydrofluoroalkanes (e.g. 1,1,1,2-tetrafluoroethane or 1,1,1,2,3,3,3-heptafluoro-n-propane) and volatile alkanes (e.g. propane or butane) and other substances which have significant vapour pressure at ambient temperature and pressure.

Furthermore the formulations of the present invention are characterised by good long-term physical and chemical stability.

### BRIEF DESCRIPTION OF THE DRAWINGS

FIG. 1 is a flow-chart showing the first stage of a method of preparing a formulation comprising 400 μg fentanyl.

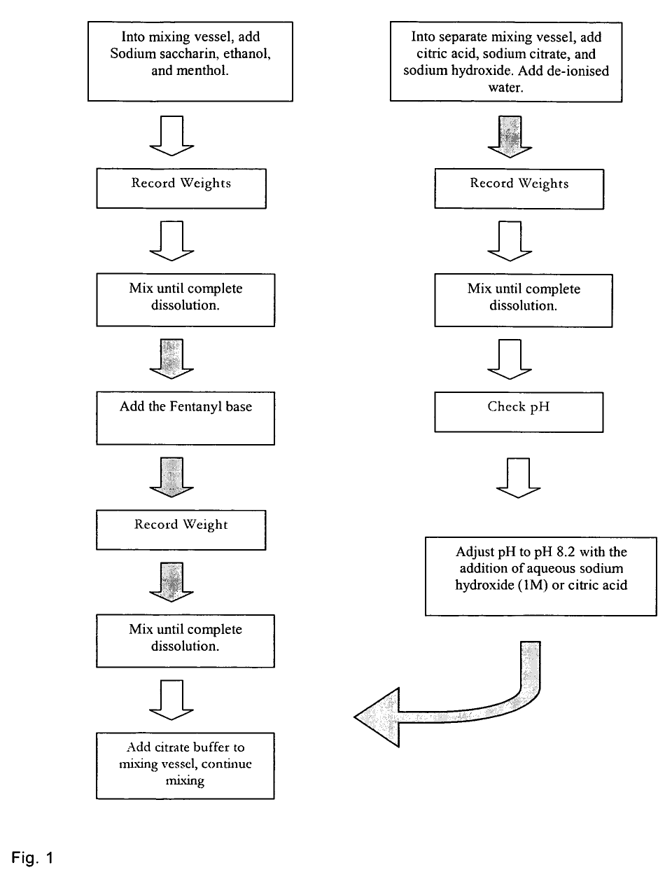

FIG. 2 is a flow-chart showing the second stage of the method.

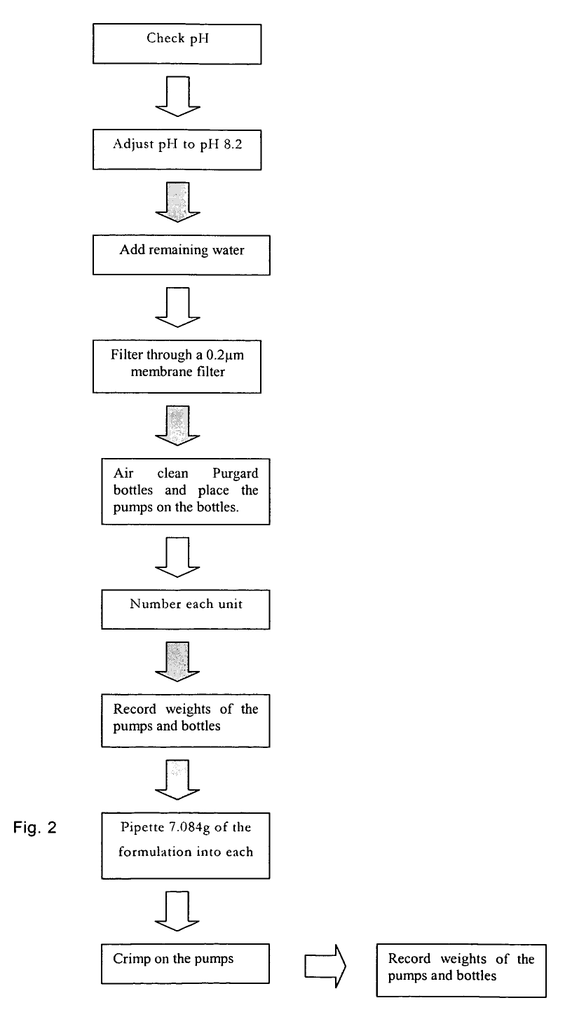

### DESCRIPTION OF THE INVENTION

The present invention provides a sublingual presentation of an opioid analgesic, such as fentanyl, which enables pain relief to be achieved very rapidly following administration of the drug.

In one embodiment of the present invention, the formulation is a solution, rather than a suspension. Whilst it is possible to spray a suspension, the fact that most suspensions settle means that the amount of active agent included in the dispensed dose will be variable and this can be highly undesirable. Although the effect of the settling of the suspension can be reduced to an extent by shaking the composition prior to spraying, some suspensions can settle very rapidly, so that there is still potential for variation of active agent content between doses.

Fentanyl may be employed in the form of a physiologically acceptable salt, which is soluble in water together with a polar organic solvent. Examples of suitable salts include hydrochloride, chloride, sulphate, tartrate and citrate. Preferably fentanyl is employed as the free base. It will nevertheless be understood that the buffer may provide some salt.

Preferably the fentanyl or physiologically acceptable salt thereof will be employed in the formulation at a concentration of 0.1 mg/ml to 10 mg/ml, preferably 0.5 mg/ml to 4.4 mg/ml (where weight is expressed as weight of fentanyl free base). More preferably, fentanyl or physiologically acceptable salt thereof will be employed in the composition at a concentration of 0.1 mg/ml to 10 mg/ml, preferably 0.5 mg/ml to 4.4 mg/ml (where weight is expressed as weight of fentanyl free base).

Examples of polar organic solvents that may be used to enhance the solubility of fentanyl, or the physiologically acceptable salt thereof in the water, include: lower alcohols (e.g. C₂₋₄ alcohols) such as ethanol; lower polyols (e.g. C₂₋₄ polyols) such as glycerol and propylene glycol; and polyethylene glycols such as PEG200 and PEG400.

Mixtures of the above substances may be used. The preferred polar organic solvent is ethanol.

In another embodiment of the present invention, the formulation does not include ethanol. Indeed, the formulation may be substantially free of any alcohol, or completely free of alcohol.

Where the composition is free of alcohol, the carrier used is preferably a polyol. The preferred polyols include propylene glycol and glycerol.

Generally it will be desired to employ the least amount of polar organic solvent necessary (or a modest excess over that necessary) to adequately solubilise the fentanyl, or physiologically acceptable salt thereof, and such that the fentanyl remains in solution under the conditions of likely usage or exposure.

The concentration of polar organic solvent is in the range preferably of between 6 and 50%, more preferably 20-45% especially 35-42%.

Preferably the water meets the USP (US Pharmacopoeia) or EP (European Pharmacopoeia) “Purified Water” standards.

It has also been found that the properties of the claimed formulations may be improved by including therein one or more additional components.

Thus, in one embodiment of the invention, the water in the formulation is present in the form of an aqueous buffer. The buffer is preferably adapted to stabilise the pH of the formulation at pH 7.4 to 8.5, preferably at pH 8.0 to 8.5, more preferably at 8.1 to 8.3, or around 8.2. At higher pH values we have found evidence that the bioavailability of the formulation is improved relative to lower pH values (e.g. nearer pH 6). Example buffer systems include sodium acetate/acetic acid, ammonium acetate/disodium edetate, boric acid/sodium hydroxide, orthophosphoric acid/sodium hydroxide, sodium hydrogen carbonate/sodium carbonate, disodium hydrogen orthophosphate/citric acid (taken from the British Pharmacopoeia). The preference is use of a citrate buffer, e.g. a buffer comprising citric acid, sodium citrate and sodium hydroxide.

The concentration of the aqueous component (water or more preferably aqueous buffer) of the formulation of the present invention is preferably 50-94%, more preferably 55-80%, and especially 58-65%.

It may be desirable to include one or more of the following components in the formulation.

1) Sweeteners, flavouring or taste-masking agents (to improve patient acceptability), for example vanilla, pineapple extract, menthol, saccharin and sodium saccharin.

2) Moisturising agents (to improve patient comfort and overcome the drying tendency of ethanol and other polar organic solvents), for example pineapple extract, lanolin, polypropylene glycol, and polyethylene glycol.

3) Penetration enhancers (to improve therapeutic effect), for example menthol.

4) Mucoadherents (in order to increase residency time on the mucosa), for example carboxyvinyl polymers, chitosans, polyacrylic acid, gelatin and polyvinyl pyrrolidone.

5) Preservatives (to improve long term resistance to microbial contamination), for example ethanol, sodium metabisulphite, benzalkonium chloride and Nipas.

6) Antioxidants, for example alkyl gallates, butylated hydroxyanisole, butylated hydroxytoluene, nordihydroguaiaretic acid, tocopherols, ascorbic acid and sodium metabisulphite.

7) Anionic surfactants, for example magnesium stearate, sodium cetostearyl sulphate, sodium lauryl sulphate, sulphated castor oil, sodium oleate, sodium stearyl fumarate and sodium tetradecyl sulphate.

8) Nonionic surfactants, for example glyceryl monostearate, macrogol cetostearyl ethers, poloxamers, polyoxyl stearates, polysorbates, sorbitan esters, sucrose esters, tyloxapol, propylene glycol monostearate, quillaia, polyoxyl caster oils, nonoxinols, lecithins and derivatives, oleic acid and derivatives, and oleyl alcohol and derivatives.

9) Foaming agents, for example alginic acid and salts, propylene glycol alginate, sodium lauryl sulphate, sodium cetostearyl sulphate, carbomers and hydroxyethylcellulose.

Some of the components proposed above may already be included in the composition of the present invention for other purposes. Suitable moisturising agents include, for example, polar organic solvents such as glycols, especially propylene glycol, and liquid polyethylene glycols, glycerol, methylcellulose, hypromellose, hydroxypropylcellulose, and many other substituted celluloses.

A versatile component, which improves the acceptability and other properties of the formulation, is menthol. Menthol, as well as flavouring the formulation, has a moisturising effect. It may also have effect, depending on its concentration, as a penetration enhancer. Preferably menthol is employed in a concentration range of 0.25% to 7.5%, although it may be yet lower.

One particular advantage of menthol is that it is compatible with fentanyl in a spray formulation unlike peppermint oil (of which menthol is one component), which causes fentanyl to degrade.

In an embodiment of the invention, the formulation contains a sweetener. The preferred sweetener is saccharin or a physiologically acceptable salt thereof such as saccharin sodium. Preferably the concentration of sodium saccharin or physiologically acceptable salt thereof is around 0.1-0.5%, e.g. around 0.28%.

Preferably the formulation contains saccharin. Surprisingly, we have found that the longer-term stability of formulations containing saccharin is better than the stability of those containing saccharin sodium.

It has been discovered that it is not generally necessary to include a preservative in the formulation when ethanol is present due to its preservative qualities.

Formulations of the invention are useful in analgesia and in the treatment of pain, e.g. the treatment of moderate to severe pain, acute pain and cancer or other breakthrough pain. A therapeutically effective amount of a formulation for the treatment of pain according to the invention may be used.

Formulations according to the invention are preferably packaged as a bulk solution containing multiple doses in a pump spray system comprising a sealed container fitted with a metering pump. Thus a sealed container may contain a plurality of doses of a formulation according to the invention. The container will preferably contain between 20 to 200 doses. Example containers are those made out of plastics, glass and metal (e.g. aluminium); glass containers are preferred. Glass containers have the advantage that the contents of the container can be seen (i.e. it is possible to determine visually when the contents are about to run out). Furthermore, glass containers are less susceptible to tampering, which is an important consideration for narcotic substances.

Preferably the glass container is coated on the exterior with a suitable moulded film of plastics material, to protect against shattering. For example, the film may be of polypropylene. The material may be coloured and contain a UV absorber. Optionally, the interior of the containers can be coated to enhance stability of the product. Coatings include polymers and lacquers but also silicone dioxide can be used to line the inside of the container with an unreactive coating.

In another embodiment, single or multiple use devices comprising a single or multiple dose of the formulation of the invention are envisaged.

The compositions of the invention are preferably dispensed using a pump spray device. Preferably, the pump consists of a metering chamber allied with efficient precompression qualities giving a high level of accuracy. Preferably, only pumps which are non-venting and are able to pass severe bacteriological challenge tests should be used. Suitable pumps include VALOIS VP7 series pumps. Preferably, the pump spray device comprising a composition of the invention is capable of administering a metered dose of the composition to the sublingual mucosa.

Also preferably, the pump spray device has features which are specifically adapted for improved sublingual delivery, as discussed in greater detail below.

When the composition is administered to the sublingual mucosa using such a device, the opioid analgesic has a surprisingly rapid onset of action. In addition, increased bioavailability is observed, compared to that observed when opioid analgesic formulations are administered sublingually using other means (such as tablets, lozenges or elixirs) or devices (such as pressurised meter dose inhaler or other propellant-driven spray devices).

When the compositions of the present invention are dispensed using a pump spray device, the composition will be dispensed at a much slower speed than is observed when compositions are dispensed using propellant-driven aerosol devices. Firstly, this will reduce the impact the composition has on the sublingual area, thereby reducing the pain which the high velocity impact can cause. This will also avoid the additional salivation which may be caused by the high velocity impact of a liquid on the sublingual mucosa. Such additional salivation can cause the administered composition to be washed away from the sublingual mucosa, preventing its absorption. Secondly, the reduced velocity of the compositions of the present invention can reduce the tendency of the dispensed compositions to form a cloud or mist which contacts areas other than the sublingual area within the mouth.

The oral mucosa can be distinguished according to important regions in the oral cavity; the floor of the mouth (sublingual), the buccal mucosa and the inner side of the lips. The sublingual mucosa is relatively permeable, giving rapid absorption of selected drug formulations. However the buccal mucosa is considerably less permeable and, therefore, not able to give rapid absorption. In an important embodiment of the present invention, the composition is administered to the majority of the area of the sublingual mucosa, preferably the composition is administered to more than 50, 60, 70, 80, 90, 95 or 98% of the area of the sublingual mucosa. This is possible when the composition is dispensed using a pump spray device at relatively slow speeds, as discussed above, and when a large enough volume of the composition is being dispensed for the desired extensive coverage.

Another aspect of the invention is a metered dose dispensing system comprising a sealed container containing a formulation of the invention fitted with a metering pump, an actuator and a channelling device. The metered dose dispensing system is preferably adapted for sublingual administration. Commercially available pump spray devices can be obtained from a limited number of suitable providers, such as, for example, Valois Pharmaceutical Division, Route des Falaises, 27100 Le Vaudreuil, France, and Bespak Europe Limited, Bergen Way, King's Lynn, Norfolk, PE30 2JJ, United Kingdom.

Suitable metering pumps include those adapted for dispensation with the container in the upright or inverted orientation. Preferably the metering chamber is adapted for dispensation with the container in the upright orientation since this facilitates administration under the tongue. Accordingly the metering chamber will be in communication with the bulk formulation by means of a dip-tube.

Example metering pumps are those manufactured by Valois and illustrated in WO01/66089.

The metering pump is preferably a non-venting type with a dip tube. Such non-venting metering pumps may have, for example, a 100 μl metering chamber capacity. The materials of construction include polypropylene and polyethylene. Suitable sealing materials, e.g. thermoplastic crimp gaskets suitable for the purpose will be employed. In addition, a suitable aluminium ferrule purposely designed for crimping on to glass containers may suitably be employed. Suitable grade stainless steel springs will preferably be adopted.

Preferably the actuator is designed to deliver a sublingually effective dose. The package may be further enhanced by the fitting of a lock-out system to promote compliance by patients.

A standard spray device used for sublingual administration will generally dispense a volume of about 50 μl of the composition upon a single actuation of the device and this volume will be administered in a localised fashion, contacting just a relatively small portion of the sublingual mucosa. In contrast, in a preferred embodiment of the present invention, a volume of 100 μl is administered in a single actuation of the pump spray device, and this dose can be applied to the majority of the area of the sublingual mucosa.

Administration of the opioid analgesic composition as a fine layer covering the majority of the sublingual area, according to the present invention, will lead to faster absorption (and therefore a faster onset of the therapeutic effect) and will result in a greater proportion of the formulation being absorbed and therefore having a therapeutic effect (i.e. greater bioavailability).

In one embodiment of the invention, the relief of acute pain is experienced very shortly after administration of a dose of opioid analgesic, such as fentanyl. Preferably, the composition has a time-to-onset-of-action of less than 30 minutes, less than 15 minutes, less than 10 minutes or less than 5 minutes, following sublingual spray administration.

Thus, compared to the known propellant-driven systems, it has been found that the pump action spray system of the present invention provides therapeutic plasma levels in a shorter time. In order for the opioid analgesic to have a pain-relieving effect, a plasma concentration of between 250 pg/ml and 2 ng/ml is required. The therapeutically effective concentrations vary between patients and it is therefore generally necessary to titrate. According to the present invention, a plasma concentration of at least 250 pg/ml may be provided within no more than 30 minutes of sublingual administration, preferably within no more than 15 minutes.

The present invention may achieve equivalent efficacious pain relief at lower maximum plasma concentrations compared with those observed as a result of intravenous administration and this, in turn, results in lower incidence of adverse side-effects. When an opioid analgesic is administered intravenously, the plasma concentration will rapidly peak, generally at a level which far exceeds what is necessary for the desired therapeutic effect. These high plasma peaks are frequently associated with severe side-effects. What is more, in order to obtain a therapeutic effect of adequate duration, it is often necessary to administer larger doses of opioid analgesics intravenously than is necessary when they are administered according to the present invention. The administration of these larger doses can also lead to adverse side-effects.

As mentioned above, the present invention also provides increased bioavailability of the opioid analgesic. In one embodiment of the invention, the compositions of the invention, or the pump spray devices comprising the compositions, provide, upon administration, a bioavailability, as determined by AUCinf (Area Under the Curve to Infinity), of no less than 60% that of intravenous administration, preferably no less than 65% of intravenous administration, more preferably no less than 70% of intravenous administration. Alternative sublingual aerosol presentations typically demonstrate lower bioavailability than this and it has been disclosed that the bioavailability of other oral transmucosal formulations presented in different delivery systems may be as low as 50% (MicroMedex HEALTHCARE Services 2001; Volume 108).

The increased bioavailability means that smaller doses can be administered whilst still achieving the same plasma concentrations and therapeutic effect, as a greater proportion of the dispensed dose is actually absorbed, reducing wastage. This has cost and safety benefits, and can once again lead to a reduced incidence of adverse side-effects.

The preferred doses according to the invention range from 100 to 3000 μg per actuation.

A further feature of the present invention, in addition to the very short speed-to-onset-of-action, is that the pain relief is maintained for at least 60 minutes. However, in some cases, it is preferable for the duration of action to be maintained throughout a period of at least 2 hours following administration of the treatment dosage. Because acute pain typically only lasts for short periods, it is often desirable for the duration of action to be maintained for at least 15 minutes but throughout a period of not greater than 2 hours. Preferably, the duration of action should be maintained over a period of 15, 20, 25, 30, 35, 40, 45, 50, 55, 65, 70, 75, 80, 90, 100, 110 or 120 minutes. Preferably, duration of action should be maintained over a period of at least 30 minutes and throughout a period of not greater than 60 minutes.

The rapid offset of the therapeutic effect observed in connection with the present invention avoids prolonged adverse events like respiratory depression, nausea and vomiting once the breakthrough pain episode has passed.

Thus, the present invention not only provides a rapid onset of the therapeutic action of the opioid analgesic, but also provides a duration-of-action which is suitable for the treatment of breakthrough pain.

In addition to the above discussed benefits of the present invention, a higher degree of patient compliance is also anticipated as a result of presenting the spray to the sublingual mucosa at lower velocities. This is because such administration results in less discomfort and surface “bounce-back”. Patient acceptability may also be improved by buffering the compositions at a pH similar to that found in oral mucosal tissue.

The compositions of the present invention are well tolerated when administered to the sensitive sublingual mucosa.

Typically a patient is treated by administration sublingually of 1 to 4 actuations, e.g. 1 or 2 actuations, from the spray pump. Another advantage of sublingual spray delivery is the ability to easily titrate patients by 1 or 2 doses as required by a single actuation. This is not the case with other forms of drug delivery (patches, lozenges, tablets, suppositories).

One of the possible methods for preparing certain formulations and filled containers according to the invention is shown in the drawings, for illustrative purposes. Other formulations of the invention may be prepared by analogous methods, or methods known to a skilled person.

Weight percentage values given herein are expressed as w/w.

The formulations and products of the invention have better physical and chemical stability, are more environmentally friendly, are more conveniently or safely administered to patients, are more conveniently or safely manufactured, are more economical to manufacture, or have other advantages relative to prior art formulations and products.

The invention will now be illustrated by reference to the following Examples.

General

Citrate buffer when employed contained:

| | |
|---|---|
| Citric acid | 2.0% |
| Sodium citrate | 1.0% |
| Sodium Hydroxide | 1.0% |
| water: | to 100% |
| pH 8.2 (adjusted with NaOH). | |

### EXAMPLE 1

Formulation (per container):

| | |
|---|---|
| Fentanyl base | 0.0280 g |
| Saccharin | 0.0177 g |
| Absolute ethanol | 2.8336 g |
| Menthol | 0.0531 g |
| Citrate buffer | 4.1516 g |

The target dose is 400 μg per actuation.

### EXAMPLE 2

Formulation (per container):

| | |
|---|---|
| Fentanyl base | 0.0280 g |
| Saccharin sodium | 0.0198 g (equivalent to 0.0177 g saccharin) |
| Absolute ethanol | 2.8336 g |
| Menthol | 0.0531 g |
| Citrate buffer | 4.1516 g |

The target dose is 400 μg per actuation of 100 μl.

### EXAMPLE 3

Formulation (per container):

| | |
|---|---|
| Fentanyl base | 0.0280 g |
| Saccharin | 0.0177 g |
| Absolute ethanol | 2.8336 g |
| Citrate buffer | 4.2047 g |

The target dose is 400 μg per actuation of 100 μl.

### EXAMPLE 4

Formulation (per container):

| | |
|---|---|
| Fentanyl base | 0.0280 g |
| Saccharin sodium | 0.0198 g (equivalent to 0.0177 g saccharin) |
| Absolute ethanol | 2.8336 g |
| Water | 4.2026 g |

The target dose is 400 μg per actuation of 100 μl.

### EXAMPLE 5

Formulation (per container):

| | |
|---|---|
| Fentanyl base | 0.0140 g |
| Saccharin sodium | 0.0198 g (equivalent to 0.0177 g saccharin) |
| Absolute ethanol | 2.8336 g |
| Menthol | 0.0531 g |
| Citrate buffer | 4.1656 g |

The target dose is 200 μg per actuation of 100 μl.
Packaging of Formulations

The example formulations may be packaged into a suitable coated glass container and fitted with a suitable non-venting metered dose pump. An actuator suitable for sublingual delivery may be fitted.

Test Data

The formulation of Example 1 was subjected to the following tests. Units were placed on stability storage at 5° C., 25° C./60% RH, 30° C./65% RH and 40° C./75% RH. For each test 3 replicates were assessed.

a) Appearance (Including Clarity).

Observation be made and the results recorded.

b) Mean Weight of Expelled Dose (Shot Weight)

Each unit will be weighed before and after test sprays. From these measurements, mean shot weight will be calculated by difference calculation

c) pH

pH is measured on a single unit at each time point at each condition. The unit is opened under controlled conditions and the pH measured by use of a pH meter.

d) Degradation Products

A sample of the formulation from each unit was taken and examined for degradation products by HPLC assay. The result was recorded as “none”, <0.1% (no identification) or percentage of identified degradant.

The results were as follows:

| | Condition | Condition | Condition | Condition |
|---|---|---|---|---|
| Test (Specification) | A | B | C | D |
| Appearance | Pass | Pass | Pass | Pass |
| (clear, no particles, | | | | |
| colourless) | | | | |
| Shotweight | Pass | Pass | Pass | Pass |
| (90-110 mg) | | | | |
| pH (7.7-8.7) | 8.2 | 8.2 | 8.2 | 8.2 |
| Degradation | ND | ND | ND | <0.1% |
| product A | | | | |
| Degradation | ND | ND | ND | 0.1-0.15% |
| product B | | | | |

The formulation of Example 2 was subjected to the same tests, with the following results:

| | Condition | Condition | Condition | Condition |
|---|---|---|---|---|
| Test | A | B | C | D |
| Appearance | Pass | Pass | Pass | Pass |
| (clear, no particles, | | | | |
| colourless) | | | | |
| Shotweight | Pass | Pass | Pass | Pass |
| (90-110 mg) | | | | |
| pH (7.7-8.7) | 8.3 | 8.3 | 8.3 | 8.3 |
| Degradation | ND | ND | ND | 0.1% |
| product A | | | | |
| Degradation | ND | ND | ND | <0.1% |
| product B | | | | |

Condition A: 2-8° C., ambient humidity
Condition B: 25° C., 60% relative humidity
Condition C: 30° C., 60% relative humidity
Condition D: 40° C., 75% relative humidity
Appearance: all samples were clear and colourless with no particles.
Shotweight: all samples were within target.
pH: stable (8.2-8.3).
Moisture content: acceptable.
Degradation products A and B: none detected.

From the test results, it was concluded that the tested formulations of the invention demonstrate excellent physical and chemical stability.

### EXAMPLE 6

A study involving a comparison between sublingual administration of formulations comprising fentanyl using a pMDI aerosol device and sublingual administration of formulations comprising fentanyl using a non-pressurised pump spray according to the invention was performed. The pMDI aerosol device used in this study was an unmodified pressurised metered dose inhaler aerosol device and was used to administer fentanyl sublingually instead of by inhalation.

The following formulations were prepared and were used to charge the containers of the dispensing devices indicated.

| Formulation 1 | | Formulation 2 | |
|---|---|---|---|
| Raw Material | g/Unit | Raw Material | g/Unit |
| Fentanyl Base | 0.0240 | Fentanyl Base | 0.0110 |
| Peppermint oil | 0.0720 | Ethanol | 2.2319 |
| Ethanol | 0.8810 | Menthol | 0.0417 |
| Propellant p134a | 5.9040 | Citrate Buffer | 3.2675 |
| | | Saccharin | 0.0139 |
| Total Unit Wt | 6.8810 | Total Unit Wt | 5.5660 |

| | | |
|---|---|---|
| Container | Purgard Schott bottle | Purgard Schott bottle |
| Valve | Bespak BK357 EPDM | Valois VP7/100D |
| Actuator | Bespak Std Button | Valois PRS102A |

Patients were treated and their plasma concentration of fentanyl measured according to the following procedure:

Blood Sampling

On the morning of Day 1 of each treatment period, subjects had an indwelling cannula inserted into a suitable forearm vein.

Blood samples (9 ml) were collected pre-dose and at the following time points following completion of the dose: 2, 5, 10, 20, 30, 40, 50, 60 and 90 minutes, and 2, 2.5, 3, 4, 5, 6, 8, 10, 12 and 24 hours.

Each sample was placed in a lithium-heparin tube and centrifuged at 3000 g for 10 minutes at 4° C., following which the plasma was separated and divided into 2 further tubes. Plasma samples were stored at −20° C., until analysis of plasma fentanyl concentration.

Plasma Assay

Plasma fentanyl concentrations were determined using a liquid chromatography-mass spectrometry/mass spectrometry (LC-MS/MS) assay with a limit of detection of 0.05 ng/ml.

Table 1 illustrates plasma profile of fentanyl uptake, in six healthy volunteers, over time using a fentanyl aerosol composition (Formulation 1), which is sublingually administered using a pMDI aerosol device.

Table 2 illustrates plasma profile of fentanyl uptake, in twelve healthy volunteers, over time using a fentanyl spray composition (Formulation 2), sublingually administered using a non-pressurised pump spray device.

TABLE 1
Pharmacokinetic parameters following sublingual administration
of 200 ug fentanyl using a pMDI aerosol device.

| Subject | Cmax (pg/ml) | Tmax (h) |
|---|---|---|
| 1 | 370 | 0.667 |
| 2 | 260 | 2 |
| 3 | 630 | 0.5 |
| 4 | 480 | 0.4166 |
| 5 | 580 | 1.5 |
| 6 | 760 | 0.667 |
| N | 6 | 6 |
| Mean | 513 | |
| SD | 181.5 | |
| Min | 260 | 0.4166 |
| Median | 530 | 0.667 |
| Max | 760 | 2 |
| Range | 500 | 1.5834 |
| CV % | 35.36 | |
| Geometric Mean | 484 | |
| CV % Geo Mean | 40.64 | |

TABLE 2
Pharmacokinetic parameters following sublingual
administration of 200 ug fentanyl administered
using a non-pressurised pump spray.

| Subject | Cmax (pg/ml) | Tmax (hr) |
|---|---|---|
| 1 | 378 | 0.333 |
| 2 | 305 | 0.5 |
| 3 | 527 | 0.5 |
| 4 | 509 | 0.5 |
| 5 | 755 | 0.333 |
| 6 | 410 | 0.667 |
| 7 | 273 | 0.5 |
| 8 | 357 | 0.333 |
| 9 | 638 | 0.333 |
| 10 | 712 | 0.333 |
| 11 | 594 | 0.667 |
| 12 | 737 | 0.833 |
| N | 12 | 12 |
| Mean | 516.3 | |
| SD | 171.79 | |
| Min | 273.0 | 0.333 |
| Median | 518.0 | 0.5 |
| Max | 755.0 | 0.833 |
| Range | 482.0 | 0.5 |
| CV % | 33.28 | |
| Geometric Mean | 488.69 | |
| CV % Geometric | 36.46 | |
| Mean | | |

The composition according to the present invention (non-pressurised pump spray) exhibits a faster onset of action, with a median Cₘₐₓ of 518.3 pg/ml and a median Tₘₐₓ of 30 mins. In contrast, the alternative composition (pMDI aerosol device) exhibits a median Cₘₐₓ of 530 pg/ml and a median Tₘₐₓ of 40 mins.

The plasma level of fentanyl required to provide the desired therapeutic effect, i.e., relief from breakthrough pain, is between 250 pg/ml and 2 ng/ml.

The plasma concentration of fentanyl following the administration of the composition according to the present invention also decreases more rapidly following the plasma concentration peak than with the alternative composition, indicating a faster offset of effect. Indeed, the plasma concentration starts to fall after just 30 minutes following administration according to the present invention. In contrast, at this point the plasma concentration of the alternative composition is still rising. What is more, the concentration also falls at a more rapid rate. The plasma concentration of fentanyl following administration according to the present invention falls to below 200 pg/ml after around 165 minutes, whilst following administration of the other composition, the plasma concentration does not fall to 15 that level until after almost 300 minutes (5 hours). This faster effect is particularly desirable when the fentanyl is being administered to treat breakthrough pain which involves episodes of relatively short duration, usually between 5 minutes and 2 hours in duration. The fast offset reduces the incidence of undesirable side-effects.

Thus, to summarise, the present invention provides relatively fast onset of action and peak plasma concentrations, as well as having an extended duration of effect as shown by the slow sloping downward curve as opposed to the relatively steep downward curve of intravenous administration. However, this duration of effect is shorter than the sublingual aerosol presentation, thereby providing benefits over the duration of pain symptoms with additional benefit of a reduced time period of side-effect.

We claim:

1. A pharmaceutical formulation having a pH of between about 7.4 and about 8.5, and comprising:
(a) fentanyl;
(b) water as carrier;
(c) a polar organic solvent in sufficient amount to enhance the solubility of the fentanyl in the water; and
(d) a buffer.

2. The formulation according to claim 1, wherein the fentanyl is present at a concentration of 0.1-10 mg/ml.

3. The formulation according to claim 1, wherein the polar organic solvent is selected from ethanol, propylene glycol, glycerol, polyethylene glycol and mixtures thereof.

4. The formulation according to claim 1, wherein the polar organic solvent is ethanol.

5. The formulation according to claim 1, wherein the polar organic solvent is present in an amount of 6-50% w/w.

6. The formulation according to claim 1, wherein the buffer is citrate buffer.

7. The formulation according to claim 1, which has pH around 8.2.

8. The formulation according to claim 1, which contains a sweetener.

9. The formulation according to claim 8, wherein the sweetener is saccharin.

10. The formulation according to claim 1, which contains menthol.

11. The formulation according to claim 1, wherein the fentanyl is introduced as the free base.

12. A metered dose dispensing system comprising a sealed container fitted with a metering pump, an actuator and a channelling device, wherein the container contains a pharmaceutical formulation having a pH of between about 7.4 and about 8.5, and comprising:
(a) fentanyl;
(b) water as carrier;
(c) a polar organic solvent in sufficient amount to enhance the solubility of the fentanyl in the water; and
(d) a buffer;
and wherein the system is adapted for sublingual administration of the formulation as a spray.

13. A pharmaceutical liquid composition comprising an opioid analgesic, the composition providing a plasma concentration of at least 250 pg/ml of opioid analgesic within a period of no more than 2 hours, following sublingual administration using a pump spray dispensing device.

14. The composition according to claim 13, wherein a plasma concentration of at least 250 pg/ml of opioid analgesic is achieved within a period of no more than 30 minutes.

15. The composition according to claim 13, wherein a plasma concentration of at least 250 pg/ml of opioid analgesic is achieved within a period of no more than 15 minutes.

16. The composition according to claim 13, wherein the opioid analgesic is fentanyl.

17. The composition according to claim 13, which is substantially free of any absorption enhancer.

18. The composition according to claim 13, which is substantially free of propellant.

19. The composition according to claim 16, wherein the fentanyl is present as the free base.

20. The composition according to claim 13, wherein the opioid analgesic is present at a concentration of 5 μg/ml to 50 mg/ml.

21. A method for providing analgesia or for the treatment of pain, which comprises administering sublingually, as a spray, a pharmaceutical liquid composition comprising an opioid analgesic, the composition providing a plasma concentration of at least 250 pg/ml of opioid analgesic within a period of no more than 2 hours, following sublingual administration using a pump spray dispensing device.

22. The method according to claim 21, wherein a volume of at least 60 μl and no more than 150 μl of the composition is administered to the sublingual area.

23. The method according to claim 21, wherein the composition has a bioavailability of more than 60% when delivered by a non-propellant pump spray device to the sublingual area.

24. The method according to claim 23, wherein the bioavailability is more than 70%.
